(19) United States
(12) Patent Application Publication
(10) Pub. No.: US 2018/0242895 A1
(43) Pub. Date: Aug. 30, 2018

(54) DEVICE AND METHOD FOR SAMPLE COLLECTION

(71) Applicant: Coyote Bioscience Yixing Co., Ltd., Wuxi (CN)

(72) Inventors: Xiang Li, Beijing (CN); Xiaobing Mu, Beijing (CN); Yuguang Han, Chengde City (CN); Huiying Feng, Beijing (CN)

(21) Appl. No.: 15/809,798

(22) Filed: Nov. 10, 2017

Related U.S. Application Data
(63) Continuation of application No. PCT/CN2015/079706, filed on May 25, 2015.

(51) Int. Cl. A61B 5/15; B01L 3/00; B65D 81/18; B01L 3/02; A61J 1/20; A61M 5/28; C12Q 1/6876

(52) U.S. Cl. CPC A61B 5/150755; C12Q 1/6806; B01L 3/5023; B65D 81/18; A61B 5/150022; A61B 5/150251; A61B 5/150343; B01L 3/022; A61J 1/2093; A61M 5/283; C12Q 1/6876; B01L 2200/025; B01L 2300/044; B01L 2300/0672; B01L 2400/0406; B01L 2400/0478; A61B 5/1405

(57) ABSTRACT

The present disclosure provides a device for collecting a bodily fluid sample of a subject for nucleic acid amplification, a method for collecting a bodily fluid sample of a subject for nucleic acid amplification, and a kit for nucleic acid amplification. The device may comprise a device body that is coupled to at least one collection channel including a collection member having an opening at an end thereof to permit flow of the bodily fluid sample from a source thereof to the collection channel. The collection channel may be substantially free of an anticoagulant. The device may further comprise a mechanical member coupled to the device body. The mechanical member may actuate the collection member to (i) extend away from the collection channel for collection of the bodily fluid sample from the source thereof, and (ii) retract into the collection channel upon collection of the sample.

### CROSS-REFERENCE

This application is a continuation of PCT Application Serial No. PCT/CN2015/079706, filed May 25, 2015, which application is herein incorporated by reference in its entirety for all purposes.

### BACKGROUND

Molecular diagnostics based on polymerase chain reaction (PCR) techniques have been widely used, e.g., in the detection of microorganisms and viruses. Samples (e.g., a blood sample) for use in laboratory testing are often obtained by way of venipuncture or through non-venous puncture, then, the samples are typically transferred to a laboratory for further processing and testing by service providers, e.g., a laboratory technician or a nurse. Samples are normally fractionated (e.g., by centrifugation), purified and processed to extract certain components or molecules therein, which may then be examined to reveal information suitable for diagnosis. The tests often involve amplifying and sequencing of nucleic acid molecules present in the samples, e.g., by PCR (e.g., qPCR).

### SUMMARY

Although there are methods and systems currently available for the collection of bodily fluid or tissue samples from a subject, recognized herein are various limitations associated with such methods and systems. For example, normally, a relatively large quantity of sample may be required to obtain reliable test results, the transportation process can be lengthy, samples can get contaminated or deteriorated in the process, it may take days or even weeks to obtain test results, and it often requires expertise and professional experience to conduct the test and/or interpret test results. Also, during the process of obtaining, transporting and testing of the samples, analyst (e.g., a nurse) can be at the risk of being infected by pathogens or viruses comprised in the sample. These may be problematic for both the subject to be tested and the analyst performing the test. Recognized herein is the need for devices and methods that enable fast, safe, and reliable molecular testing.

The present disclosure provides devices and methods for collecting a sample from a subject in a fast and simple manner, which makes it possible to perform on-site molecular diagnosis quickly and easily, while generating reliable result.

An aspect of the present disclosure provides a device for collecting a bodily fluid sample of a subject for nucleic acid amplification. The device comprises: a device body that is coupled to at least one collection channel including a collection member having an opening at an end thereof to permit flow of the bodily fluid sample from a source of the bodily fluid sample to the collection channel, wherein the collection channel is substantially free of an anticoagulant; and a mechanical member coupled to the device body, wherein the mechanical member actuates the collection member to (i) extend away from the collection channel for collection of the bodily fluid sample from the source of the sample, and (ii) retract into the collection channel upon collection of the bodily fluid sample from the source. The mechanical member may actuate the collection member to alternately extend away from the collection channel and retract into the collection channel.

In some embodiments, the bodily fluid sample is a blood sample. In some embodiments, the blood sample is a whole blood sample.

In some embodiments, the collection channel is substantially contained in the body. In some embodiments, the mechanical member is substantially contained in the body.

In some embodiments, the device further comprises a collection vessel capable of being in fluid communication with the collection channel, wherein the collection vessel includes reagents necessary for nucleic acid amplification. In some embodiments, the collection vessel is substantially free of an anticoagulant.

In some embodiments, the reagents include one or more primers and a polymerizing enzyme. In some embodiments, the reagents include Mg or Mn ions. In some embodiments, the one or more primers have sequences that are selected to assay for a presence of an infectious disease in the subject.

In some embodiments, the collection vessel is adapted to stably store the mixture for a time period of at least about 5 minutes. In some embodiments, the bodily fluid sample has a volume that is less than about 1 mL.

In some embodiments, the source is a pool of the bodily fluid sample in a storage vessel. In some embodiments, the source is a tissue of the subject that is accessible through a puncture in the tissue.

In some embodiments, the end includes a needle that is adapted to generate the puncture.

In some embodiments, the collection member includes a needle adjacent to a needle cover, wherein the needle includes the opening, and wherein the needle is actuatable to penetrate the needle cover for collection of the bodily fluid sample from the source.

In some embodiments, the mechanical member is a button. In some embodiments, the mechanical member is spring loaded. In some embodiments, the mechanical member is depressable at a first position and further depressable at a second position, wherein depression at the first position causes the collection member to extend away from the collection channel for collection of the bodily fluid sample from the source of the sample, and wherein depression at the second position causes the mixture to flow from the collection channel to the opening. In some embodiments, depression at the second position causes the collection channel to extend away from a body of the collection device, wherein the body includes the mechanical member.

In some embodiments, the collection vessel includes identifying information of the subject. In some embodiments, the identifying information is anonymous. In some embodiments, the identifying information is on a barcode. In some embodiments, the identifying information is in a radio-frequency identification (RFID) tag.

Another aspect of the present disclosure provides a method for collecting a bodily fluid sample of a subject. The method comprises: providing a sample collection device comprising at least one collection channel, wherein the collection channel includes a collection member having an opening at an end thereof to permit flow of the bodily fluid sample from a source of the bodily fluid sample to the collection channel; positioning the opening of the collection member adjacent to the source of the sample; collecting the bodily fluid sample from the source to the collection channel upon flow of the bodily fluid sample from the source through the opening into the collection channel; and depositing the bodily fluid sample collected in (c) from the collection channel through the opening into a collection vessel that includes reagents necessary for nucleic acid amplification, thereby providing a mixture having the bodily fluid sample and the reagents.

In some embodiments, the bodily fluid sample is a blood sample. In some embodiments, the blood sample is a whole blood sample.

In some embodiments, the method further comprises performing the nucleic acid amplification using the bodily fluid sample deposited in the collection vessel. In some embodiments, the collection vessel is part of the collection device. In some embodiments, the collection vessel is substantially free of an anticoagulant. In some embodiments, the collection channel is substantially free of an anticoagulant.

In some embodiments, the reagents include one or more primers and a polymerizing enzyme. In some embodiments, the reagents include Mg or Mn ions. In some embodiments, the one or more primers have sequences that are selected to assay for a presence of an infectious disease in the subject.

In some embodiments, the collection vessel is adapted to stably store the mixture for a time period of at least about 5 minutes.

In some embodiments, steps (a)-(d) are performed in a time period less than about 10 minutes. In some embodiments, the time period is less than about 1 minute. In some embodiments, the time period is less than about 30 seconds.

In some embodiments, the bodily fluid sample has a volume that is less than about 1 mL.

In some embodiments, the source is a pool of the bodily fluid sample in a storage vessel. In some embodiments, the source is a tissue of the subject that is accessible through a puncture in the tissue.

In some embodiments, the collection member includes a needle that is adapted to generate the puncture. In some embodiments, the collection member includes a needle adjacent to a needle cover, wherein the needle includes the opening, and wherein the needle is actuatable to penetrate the needle cover for collection of the bodily fluid sample from the source.

In some embodiments, the sample collection device includes a mechanical member that actuates the collection member to (i) extend away from the collection channel for collection of the bodily fluid sample from the source of the sample, and (ii) retract into the collection channel upon collection of the bodily fluid sample from the source. The mechanical member may actuate the collection member to alternately extend away from the collection channel and retract into the collection channel.

In some embodiments, the mechanical member is a button.

In some embodiments, the mechanical member is depressable at a first position and further depressable at a second position, wherein depression at the first position causes the collection member to extend away from the collection channel for collection of the bodily fluid sample from the source of the sample, and wherein depression at the second position causes the sample to flow from the collection channel to the opening. In some embodiments, depression at the second position causes the collection channel to extend away from a body of the collection device, wherein the body includes the mechanical member.

In some embodiments, the mechanical member is (i) depressable from a first position towards a second position to extend the collection member away from the collection channel, and (ii) extendable from the second position towards the first position to retract the collection member into the collection channel upon collection of the sample.

Another aspect of the present disclosure provides a kit for nucleic acid amplification. The kit comprises: a collection vessel including reagents necessary for nucleic acid amplification, wherein the collection vessel is substantially free of an anticoagulant; and instructions that permit a user to use a collection device to (i) collect a bodily fluid sample from a source of the sample, and (ii) deposit the bodily fluid sample into the collection vessel to provide a mixture comprising the bodily fluid sample and the reagents for nucleic acid amplification in a time period that is less than about 1 minute.

In some embodiments, the instructions permit the user to perform nucleic acid amplification using the sample.

In some embodiments, the time period is less than about 30 seconds.

In some embodiments, the kit further includes the collection device. In some embodiments, the collection device includes at least one collection channel that includes a collection member having an opening at an end thereof to permit flow of the bodily fluid sample from the source of the bodily fluid sample to the collection channel.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIGS. 1A-1D demonstrate a workflow for sample collection using a sample collection device of the present disclosure.

FIG. 2 shows enlarged views of part of a sample collection device of the present disclosure.

FIGS. 2A-2C illustrates change of needle (205) position during the sample collection process.

FIGS. 3A-3D demonstrate a workflow for sample collection using another sample collection device of the present disclosure.

FIG. 4 illustrates a reaction vessel.

FIG. 5 illustrates a system comprising a collection device for performing sample collection and sample examination.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “substantial”, as used herein, generally refers to more than a minimal or insignificant amount; and “substantially” generally refers to more than minimally or insignificantly. The term “substantially free of,” as used herein with respect to an amount, quantity or concentration of a substance, generally means that that there is less than about 10% (v/v), less than about 5% (v/v), less than about 4% (v/v), less than about 3% (v/v), less than about 2% (v/v), less than about 1% (v/v), less than about 0.1% (v/v), less than about 0.01% (v/v), less than about 0.001% (v/v) ,or less than about 0.0001% (v/v) of the substance in a mixture or a device, or a component of the device.

The term “sample,” as used herein, generally refers to a tissue or a bodily fluid sample. For example, a sample can be but is not limited to a blood sample, or a portion thereof. A sample may contain or be suspected of containing a nucleic acid molecule. The sample can include cellular material. The sample can include nucleic acid material, such as deoxyribonucleic acid (DNA) or ribonucleic acid (RNA). For example, a subject sample can be a biological sample containing one or more nucleic acid molecules. The biological sample can be obtained or obtainable (e.g., extracted or isolated) from a bodily sample of a subject that can be selected from blood (e.g., whole blood), plasma, serum, urine, saliva, mucosal excretions, sputum, stool and tears. The bodily sample can be a fluid or tissue sample (e.g., skin sample) of the subject. In some examples, the sample is obtained from a cell-free bodily fluid of the subject, e.g., whole blood. In such instance, the sample can include cell-free DNA and/or cell-free RNA. In some other examples, the sample is an environmental sample (e.g., soil, waste, ambient air and etc.), industrial sample (e.g., samples from any industrial processes), and food samples (e.g., dairy products, vegetable products, and meat products).

A sample may be of any suitable size or volume. In some examples, a small volume comprises no more than about 5 mL; no more than about 4 mL; no more than about 3 mL; no more than about 2 mL; no more than about 1 mL; no more than about 500 μL; no more than about 250 μL; no more than about 100 μL; no more than about 75 μL; no more than about 50 μL; no more than about 35 μL; no more than about 25 μL; no more than about 20 μL; no more than about 15 μL; no more than about 10 μL; no more than about 8 μL; no more than about 6 μL; no more than about 5 μL; no more than about 4 μL; no more than about 3 μL; no more than about 2 μL; no more than about 1 μL; no more than about 0.8 μL; no more than about 0.5 μL; no more than about 0.3 μL; no more than about 0.2 μL; no more than about 0.1 μL; no more than about 0.05 μL; or no more than about 0.01 μL.

The term “point of care,” as used herein, generally refers to locations where a subject may be cared for (e.g., by testing, monitoring, treatment, diagnosis, guidance, sample collection, identification (ID) verification, medical services, non-medical services, etc.), and may include but not limited to, a subject's home, a subject's business, the location of a healthcare provider (e.g., doctor), hospitals, emergency rooms, operating rooms, clinics, health care professionals' offices, laboratories, retailers—e.g., pharmacies (e.g., retail pharmacy, clinical pharmacy, hospital pharmacy), drugstores, supermarkets, grocers, etc.—transportation vehicles (e.g., car, boat, truck, bus, airplane, motorcycle, ambulance, mobile unit, fire engine/truck, emergency vehicle, law enforcement vehicle, police car, or other vehicle configured to transport a subject from one point to another, etc.), traveling medical care units, mobile units, schools, day-care centers, security screening locations, combat locations, health assisted living residences, government offices, office buildings, tents, sample acquisition sites (e.g., blood collection centers), or any other point of care location described elsewhere in the present application.

The term “bodily fluid”, as used herein, generally refers to any fluid obtainable from a subject. A bodily fluid may include but not limited to, e.g., blood, urine, saliva, tears, sweat, a bodily secretion, a bodily excretion, or any other fluid originating in or obtainable from a subject. In particular, bodily fluids include but not limited to blood, serum, plasma, bone marrow, saliva, urine, gastric fluid, spinal fluid, tears, stool, mucus, sweat, earwax, oil, glandular secretions, cerebral spinal fluid, semen, vaginal fluid, interstitial fluids derived from tumorous tissue, ocular fluids, placental fluid, amniotic fluid, cord blood, lymphatic fluids, cavity fluids, sputum, pus, meconium, breast milk and/or other secretions or excretions.

As used herein, a “collection member” can be disposable, e.g., it can be used once and disposed. A collection member may also comprise one or more disposable components, wherein each of said components may be used once and disposed. Alternatively or in addition, a collection member may be reusable or may comprise one or more reusable components, which for example may be reused any number of times.

As used herein, a “collection channel” may be capable of receiving one or more types of sample. For example, collection channel may be capable of receiving two different types of bodily fluid sample (e.g., blood, tears).

The term “needle”, as used herein, generally refers to any article capable of penetrating a tissue or tissue surface of a subject, thereby introducing material into or removing material from the said tissue. In some embodiments, a needle can be a sharp-pointed slender instrument.

The term “button”, as used herein, generally refers to a mechanical component that can be compressed or depressed to various positions and/or levels. A button can be depressable and extendable. A button can be of any form or shape suitable for being compressed or depressed to various positions and/or levels, e.g., cylindrical, cubical, bar-shaped, rod-shaped, etc. A button of the present disclosure can be configured to drive the movement of a collection channel and/or a collection member. A button can also be configured to initiate or drive the movement of a sample into or out of a collection channel.

The term “kit”, as used herein, generally refers to a combination of two or more components, wherein said two or more components can be comprised in a single package or container. Alternatively, said two or more components can be separately comprised in two or more independent packages or containers.

The term “membrane,” as used herein, generally refers to a structure that separates at least two volumes, or that separates a volume from the external environment. A membrane may be a synthetic membrane, e.g., a membrane formed of a solid state material (e.g., semiconductor, metal, semi-metal or non-metal) or polymeric material. For example, a membrane can be formed by an opaque, transparent, or translucent material sealing a collection vessel and separating it from the external environment.

The term “nucleic acid,” as used herein, generally refers to a molecule comprising one or more nucleic acid subunits. A nucleic acid may include one or more subunits selected from adenosine (A), cytosine (C), guanine (G), thymine (T) and uracil (U), or variants thereof. A nucleotide can include A, C, G, T or U, or variants thereof including but not limited to peptide nucleic acid (PNA). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a nucleic acid is deoxyribonucleic acid (DNA) or ribonucleic acid (RNA), or derivatives thereof. A nucleic acid may be single-stranded or double stranded. A nucleic acid may comprise one or more modified nucleotides, e.g., methylated nucleotides and nucleotide analogs.

The term “polymerase,” as used herein, generally refers to any enzyme capable of catalyzing a polymerization reaction. Examples of polymerases include, without limitation, a nucleic acid polymerase, a transcriptase or a ligase. A polymerase can be a polymerization enzyme or a polymerizing enzyme.

The term “subject,” as used herein, generally refers to an animal or other organism, e.g., a mammalian species (e.g., human), avian (e.g., bird) species, or plant. Mammals include, but are not limited to, murines, simians, humans, farm animals, sport animals, and pets. A subject can be an individual that has or is suspected of having a disease or a pre-disposition to the disease, or an individual that is in need of therapy or suspected of needing therapy. A subject can be a patient.

As used herein, the term “anticoagulant” is an agent capable of maintaining a sample (e.g., a blood sample) in liquid form. An anticoagulant can be an anti-coagulating agent, such as, for example, heparin (e.g., lithium heparin or sodium heparin) or ethylenediaminetetraacetic acid (EDTA), in some cases integrated with external peripherals for integrated tests or services.

The present disclosure provides devices, methods and systems for obtaining, processing and analyzing a sample. Various aspects of the devices, systems and methods described herein may be applied to any of the particular devices, systems and methods set forth below. Devices, systems and methods provided herein may be applied as a standalone device, system or method, or as part of an integrated system, e.g., in a system involving point of care services.

### Device for Collecting a Sample of a Subject for Nucleic Acid Amplification

An aspect of the present disclosure provides a device for collecting a sample (e.g., a bodily fluid sample, e.g., a blood sample) of a subject for nucleic acid amplification. The device may comprise a device body that is coupled to at least one collection channel. The collection channel may include a collection member having an opening at an end thereof to permit flow of the sample.

In some embodiments, the system may include external amplification and/or sequencing devices (e.g., a PCR machine) or be source of the sample to the collection channel. The collection channel can be substantially free of an anticoagulant. The device can further include a mechanical member coupled to the device body. The mechanical member may be configured to actuate the collection member to (i) extend away from the collection channel for collection of the sample from the source thereof, and/or (ii) retract into the collection channel upon collection of the sample from its source. In some cases such actuation is upon engagement of the mechanical member by a user of the device, e.g., the subject. The mechanical member may actuate the collection member to alternately extend away from the collection channel and retract into the collection channel. The mechanical member may actuate the collection member to alternately and sequentially extend away from the collection channel and retract into the collection channel, or vice versa.

The device can further include a cover positioned at the end of the collection channel comprising the collection member, wherein the cover is configured to prevent the opening and the collection member from being exposed to the environment and to keep them clean. The cover may further comprise a retaining member (e.g., a sheet apparatus comprising a sample retaining card, e.g., a blood card) on the side of the cover facing the subject (e.g., a finger of the subject). During the retraction of the collection member into the collection channel after sampling, a least a substantial part of the sample (e.g., blood) carried on the collection member may be retained (e.g., absorbed) by the retaining member. If desired, the retaining member may be used for further detection, e.g., analyzing certain components or characteristics of the sample retained.

The cover may be fitted over a portion of the collection channel and/or a portion of the collection member. The cover may also be fitted within a portion of the device body. The cover may be formed from an opaque, transparent, or translucent material. The cover may also be formed from a material penetrable with a collection member (e.g., a needle) and/or a collection channel (e.g., a capillary).

The device body may comprise one or more flanges attached to a portion thereof (e.g., an end or one or more sides thereof) adjacent to the mechanical member. The one or more flanges may be present in pairs at symmetrical positions of the device body. Each of the one or more flanges may comprise at least a first contacting element and a second contacting element. The mechanical member may comprise along its length at least a first protruding element. The first contacting element may be configured to engage and be in contact with the first protruding element. The collection channel may comprise on its outer surface (i.e., the surface facing the inner wall of the device body) at least a second protruding element, wherein the second contacting element may be configured to engage and be in contact with the second protruding element.

Each of the protruding elements may be present symmetrically in pairs, with the longitudinal axis of the mechanical member and/or the collection channel to be symmetry axis. In some embodiments, the first and the second protruding element and may be positioned perpendicular to the longitudinal axis of the mechanical member and the collection channel, respectively. In some embodiments, comparing to the first protruding element, the second protruding element is located at a position closer to the opening of the collection channel, and the second protruding element is longer than the first protruding element.

In further embodiments, comparing to the first protruding element, the second protruding element is located at a position closer to the opening of the collection channel, and the second protruding element protrudes further away than the first protruding element from their respective attaching point to the mechanical member or the collection channel.

The sample can be a tissue or a bodily fluid sample, or a fraction thereof. In some cases, the sample is a “bodily fluid” sample, which may include but not limited to blood, urine, saliva, tears, sweat, a bodily secretion, a bodily excretion, or any other fluid originating in or obtainable from a subject. In particular, the sample may include but not limited to blood, serum, plasma, bone marrow, saliva, urine, gastric fluid, spinal fluid, tears, stool, mucus, sweat, earwax, oil, glandular secretions, cerebral spinal fluid, semen, vaginal fluid, interstitial fluids derived from tumorous tissue, ocular fluids, placental fluid, amniotic fluid, cord blood, lymphatic fluids, cavity fluids, sputum, pus, meconium, breast milk and/or other secretions or excretions. For example, a sample can be a blood sample, or a portion thereof, which may include but not limited to a whole blood sample, a sample comprising red blood cells, plasma sample, serum sample, buffy coat sample, a sample comprising white blood cells, etc. The blood sample can be obtained directly from the subject, e.g., the sample can be analyzed or tested (e.g., by amplification or sequencing) without further processing (e.g., by centrifugation, purification, etc.).

The collection channel can be substantially contained in the device body. In certain cases, the mechanical member can be substantially contained in the device body.

The device may further comprise a collection vessel capable of being in fluid communication with the collection channel, wherein the collection vessel may include reagents necessary for nucleic acid amplification. Thus, the sample may be deposited from the collection channel through the opening of the collection member into the collection vessel, thereby forming a mixture comprising the sample and the reagents. In some embodiments, the collection vessel can be substantially free of an anticoagulant. The reagents comprised in the collection vessel can include, but not limited to, one or more primers and one or more polymerizing enzymes. In certain cases, the reagents may comprise Mg or Mn ions.

The one or more primers may have sequences that are selected to assay for a presence or quantity of an infectious agent in said subject. The infectious agent may be associated with a disease that the subject is having, being suspected of having or at the risk of having. In some embodiments, the disease may be associated with a virus e.g., an RNA virus or a DNA virus. For example, the virus can be selected from the group consisting of human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, and Varicella virus. In some embodiments, the influenza virus can be selected from the group consisting of H1N1 virus, H3N2 virus, H7N9 virus and H5N1 virus. In some embodiments, the adenovirus may be adenovirus type 55 (ADV55) or adenovirus type 7 (ADV7). In some embodiments, the hepatitis C virus may be armored RNA-hepatitis C virus (RNA-HCV). In some embodiments, the disease may be associated with a pathogenic bacterium (e.g., Mycobacterium tuberculosis) or a pathogenic protozoan (e.g., Plasmodium).

The collection vessel can be adapted to stably store the mixture comprising the sample and the reagents for a time period of at least about 5 minutes. In some embodiments, the collection vessel may be adapted to stably store the mixture for a time period of at least about 1 hour, 2 hours, 3 hours, 4 hours, 5 hours, 6 hours, 12 hours, 1 day, 2 days, 3 days, 4 days, 5 days, 6 days, 1 week, 2 weeks, 3 weeks, or 1 month.

The sample may be a bodily fluid sample having a volume of no more than about 5 mL; no more than about 4 mL; no more than about 3 mL; no more than about 2 mL; no more than about 1 mL; no more than about 500 μL; no more than about 250 μL; no more than about 100 μL; no more than about 75 μL; no more than about 50 μL; no more than about 35 μL; no more than about 25 μL; no more than about 20 μL; no more than about 15 μL; no more than about 10 μL; no more than about 8 μL; no more than about 6 μL; no more than about 5 μL; no more than about 4 μL; no more than about 3 μL; no more than about 2 μL; no more than about 1 μL; no more than about 0.8 μL; no more than about 0.5 μL; no more than about 0.3 μL; no more than about 0.2 μL; no more than about 0.1 μL; no more than about 0.05 μL; or no more than about 0.01 μL. For example, the sample can have a volume of about 0.01 μL to about 5 mL, about 0.01 μL to about 4 mL, about 0.01 μL to about 3 mL, about 0.01 μL to about 2 mL, about 0.01 μL to about 1 mL, about 0.01 μL to about 0.5 μL, about 0.01 μL to about 0.4 μL, about 0.01 μL to about 0.3 μL, about 0.01 μL to about 0.2 μL, about 0.01 μL to about 0.1 μL, about 0.01 μL to about 0.05 μL.

The source of the sample can be a pool of the sample in a storage vessel. The source may also be a tissue of the subject that is accessible through a puncture in the tissue.

The end of the collection member may include a needle that is adapted to generate a puncture in the tissue. In some cases, the collection member can include a needle adjacent to a needle cover, the needle may include an opening, and the needle is actuatable to penetrate the needle cover for collection of sample from the sample source. In some embodiments, the mechanical member can be a button. In some cases, the mechanical member can be spring loaded. In further embodiments, the mechanical member can be depressible at a first position and further depressible at a second position. Depression at the first position may cause the collection member to extend away from the collection channel for collection of the sample from a source thereof. Depression at the second position may cause the sample to flow from the collection channel to the opening. In some embodiments, depression at the second position may cause the collection channel to extend away from the body of the collection device, wherein the body includes the mechanical member. The collection vessel may include identifying information of the subject tested. The identifying information can be anonymous. In some cases, the identifying information can be on a barcode. In further embodiments, the identifying information can be in a radio-frequency identification (RFID) tag. In some embodiments, the first position and the second position may be the same position. In further embodiments, the first position and the second position may be different positions

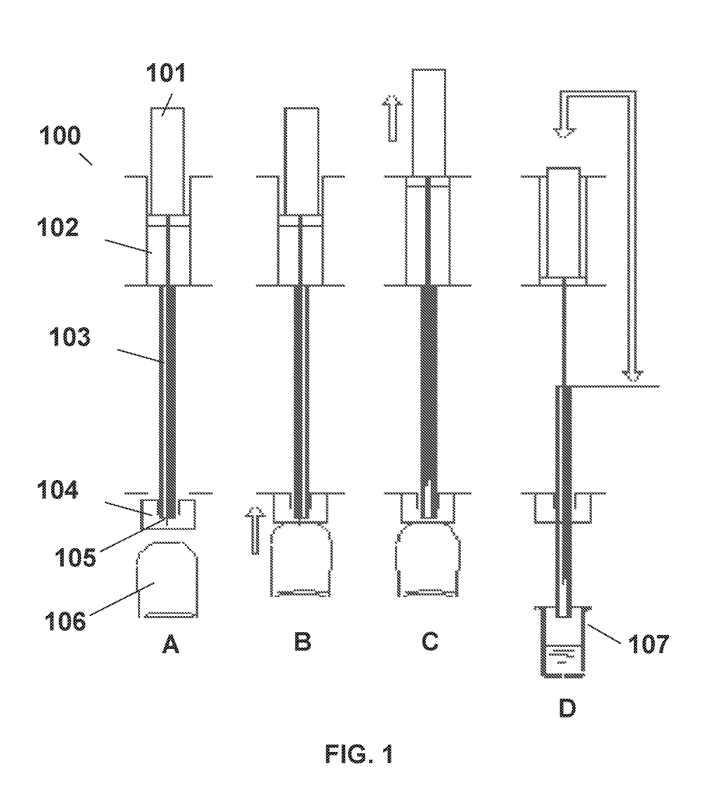

With reference to FIGS. 1A-1D, a sample collection device 100 is shown. The sample collection device 100 may include a collection device body 102, a mechanical member 101, and at least one collection channel 103 having an opening at an end thereof. The collection channel may comprise a collection member 105 at or adjacent to the end having the opening. In certain cases, the collection channel may be substantially free of any anticoagulant.

In some instances, a cover 104 may be provided. The cover 104 may be positioned at the end of the collection channel comprising the collection member, wherein the cover is configured to prevent the opening and the collection member from being exposed to the environment and to keep them clean. For example, when the sample is blood sample, the cover may be configured in an appropriate shape and position capable of protecting the opening and the collection member, keeping them clean, and covering up the bloody tip of the collection member after sampling. In one non-limiting example, the cover 104 may be fitted over a portion of the collection channel 103 or be fitted over the collection member 105. The cover 104 may be detachable from the collection channel 103. In some instances, the cover 104 may be completely separable from the collection channel 103, or may retain a portion that is connected to the collection channel 103, e.g., but not limited to being hinged or otherwise linked to the collection channel. The cover 104 may cover a portion of the collection channel 103 containing an opening at an end thereof. The cover 104 may prevent material, e.g., air, fluid, or particulates, from entering the channels, when the cover is in place. The cover 104 may attach to the collection channel 103 using any technique known or later developed in the art. For instance, the cover may be snap fit, twist on, friction-fit, clamp on, have magnetic portions, tie in, utilize elastic portions, and/or may removably connect to the collection channel. The cover may also be directly or indirectly connected to the collection channel, e.g., through an intermediate material positioned between the collection channel and the cover. The cover may form a fluid-tight seal with the collection channel. The cover may be formed from an opaque, transparent, or translucent material. The cover may be formed from a material penetrable with a collection member (e.g., a needle) and/or a collection channel (e.g., a capillary).

The collection channel may be any form of pathways able to transport and store (at least transiently) a sample, such a bodily fluid sample, e.g., blood. The collection channel may have any shape or size, some embodiments are configured such that the channel exhibits a capillary action when in contact with sample fluid. In some instances, the channel may have a cross-sectional area of less than or equal to about 10 mm², 7 mm², 5 mm², 4 mm², 3 mm², 2.5 mm², 2 mm², 1.5 mm², 1 mm², 0.8 mm², 0.5 mm², 0.3 mm², or 0.1 mm². The cross-sectional size may remain the same or may vary along the length. Some embodiments may tailor for greater force along a certain length and then less in a different length. The cross-sectional shape may remain the same or may vary along the length. Some channels are straight in configuration. Some embodiments may have curved or other shaped path shapes alone or in combination with straight portions. Some may have different orientations relative to the device body 102. For example, when the device is held substantially horizontally, one or more channels may slope downward, slope upward, or not slope at all as it carries fluid away from the initial collection point on the device.

The mechanical member 101 may be coupled to the device body 102 and may be configured to actuate the collection member 105 to extend away from the collection channel for collection of a sample from the source thereof. The collection member 105 may also be configured to retract into the collection channel upon collection of the sample from the source thereof. The mechanical member may be of any shape or form suitable for being engaged to apply a pressure and actuate the collection member. In some embodiments, the mechanical member 101 may comprise a spring or any other component capable of providing positive pressure. In the present specific embodiment, the mechanical member 101 is in the form of a button, which can either be depressed towards the other end of the device 100 (as shown in FIG. 1D) or be pushed up in the opposite direction (as shown in FIG. 1C).

In the present embodiment, the mechanical member 101 can be used to join the device body 102, the collection channel 103, the collection member 105 and/or the cover 104 to form an integrated device. It should be understood that although the mechanical member 101, the device body 102, the collection channel 103, the collection member 105 and the cover 104 are recited as separate parts, one or more of those parts may be integrally formed to simplify manufacturing and such integration is not excluded herein.

FIG. 1A shows that a subject aligns his/her finger 106 with the cover 104. As demonstrated in FIG. 1A, the mechanical member 101 of the device 100 is depressed at a first position, causing the collection member 105 to extend away from the collection channel 103 for collection of a sample. FIG. 1B shows that the finger moves towards the collection member, thereby also pressing the cover towards the collection member. As a result, the collection member (e.g., comprising a needle) may penetrate through the cover and may be exposed. Then, the exposed collection member may contact with and prick the finger, thereby permitting the sample (e.g., blood) to flow out of the finger. FIG. 1C shows that the collection member may then retract into the collection channel, which can be achieved by, e.g., a pressure created by the finger while pressing against the collection member, thereby the sample may enter into the collection channel (e.g., a capillary) through the opening at the end of the collection channel. Meanwhile, the mechanical member may be pushed away in a direction opposite to the opening of the collection channel. FIG. 1D shows that when sufficient sample has been collected in the collection channel, a positive pressure may be applied to the mechanical member, which in turn may actuate a portion of the collection channel covered by the cover and/or device body to protrude out of the cover. If desired, a further positive pressure may be applied to the mechanical member, thereby causing the sample collected to flow from the collection channel to the opening.

The sample may flow into a collection vessel 107, which may be capable of being in fluid communication with the collection channel. The collection vessel may comprise reagents necessary for nucleic acid amplification. Thus, the sample from the collection channel and reagents comprised in the collection vessel may form a reaction mixture. In some cases, the collection vessel may be substantially free of any anticoagulant. The reagents necessary for nucleic acid amplification may include one or more primers (e.g., primers specific for amplifying certain target nucleic acids), it may also include a polymerizing enzyme. The reagents may further include Mg or Mn ions. The primers may have sequences selected to assay for a presence of an infectious disease in the subject. For example, the primers may have sequences selected to assay for the presence and/or amount of one or more pathogens, including but not limited to H1N1 virus, H3N2 virus, H7N9 virus and H5N1 virus, adenovirus type 55 (ADV55) or adenovirus type 7 (ADV7, armored RNA-hepatitis C virus (RNA-HCV), a pathogenic bacterium (e.g., Mycobacterium tuberculosis) or a pathogenic protozoan (e.g., Plasmodium).

The reaction mixture can include reagents necessary to complete nucleic acid amplification (e.g., DNA amplification, RNA amplification), with non-limiting examples of such reagents including primer sets having specificity for target RNA or target DNA, DNA produced from reverse transcription of RNA, a DNA polymerase, a reverse transcriptase (e.g., for reverse transcription of RNA), suitable buffers (including zwitterionic buffers), co-factors (e.g., divalent and monovalent cations), dNTPs, and other enzymes (e.g., uracil-DNA glycosylase (UNG)), etc). In some cases, reaction mixtures can also comprise one or more reporter agents. The reaction mixture can also include an enzyme that is suitable to facilitate nucleic acid amplification, e.g., a polymerizing enzyme (also “polymerase” herein). The polymerase can be a DNA polymerase for amplifying DNA. Any suitable DNA polymerase may be used, including commercially available DNA polymerases. The DNA polymerase can be capable of incorporating nucleotides to a strand of DNA in a template bound fashion. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at 94° C.-95° C. for 2 minutes to 10 minutes may be required, which may change the thermal profile based on different polymerases.

In some cases, a DNA sample can be generated from an RNA sample. This can be achieved using reverse transcriptase, which can include an enzyme that is capable of incorporating nucleotides to a strand of DNA, when bound to an RNA template. Any suitable reverse transcriptase may be used. Non-limiting examples of reverse transcriptases include HIV-1 reverse transcriptase, M-MLV reverse transcriptase, AMV reverse transcriptase, telomerase reverse transcriptase, and variants, modified products and derivatives thereof.

Nucleic acid amplification reaction can include one or more primer extension reactions to generate amplified product(s). In PCR, for example, a primer extension reaction can include a cycle of incubating a reaction mixture at a denaturation temperature for a denaturation duration and incubating a reaction mixture at an elongation temperature for an elongation duration. Denaturation temperatures may vary depending upon, e.g., the particular biological sample analyzed, the particular source of target nucleic acid (e.g., viral particle, bacteria) in the biological sample, the reagents used, and/or the desired reaction conditions. For example, a denaturation temperature may be from about 80° C. to about 110° C. In some examples, a denaturation temperature may be from about 90° C. to about 100° C. In some examples, a denaturation temperature may be from about 90° C. to about 97° C. In some examples, a denaturation temperature may be from about 92° C. to about 95° C. In still other examples, a denaturation temperature may be at least about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

As an alternative, in isothermal amplification, the temperature can be fixed (i.e., maintained constant and not cycled), and amplification product(s) can be generated using a primer set and a polymerase with high strand displacement activity in addition to a replication activity. An example of a polymerase that may be suitable for use in isothermal amplification is Bst polymerase. The temperature can be fixed between about 50° C. and 80° C., or 60° C. and 65° C. In loop mediated isothermal amplification (LAMP), e.g., a template nucleic acid molecule can be amplified using a polymerase and a primer set having at least 2, 3, 4, or 5 primers.

The amplification of the template nucleic acid molecule and detection of the target nucleic acid molecule can be performed in the same system, e.g., a vessel. In some cases, the system is a tube that is configured for nucleic acid amplification, e.g., an eppendorf PCR tube.

The collection vessel 107 may include identifying information of the subject. The identifying information may be anonymous, it may also be on a barcode or in a radio-frequency identification (RFID) tag. The barcode can include a string of characters, e.g., letters and/or numbers.

In some embodiments, the collection vessel 107 may be sealed with a membrane (e.g., a diaphragm) before being in fluid communication with the collection channel. The membrane may be a synthetic membrane, e.g., a membrane formed of a solid state material (e.g., semiconductor, metal, semi-metal or non-metal) or polymeric material. For example, a membrane can be formed by an opaque, transparent, or translucent material sealing a collection vessel and separating it from the external environment. Thus, when sufficient sample has been collected in the collection channel, a positive pressure may be applied to the mechanical member, which in turn may actuate a portion of the collection channel covered by the cover and/or device body to protrude out of the cover, meanwhile, the collection member (e.g., including a needle) may be simultaneously actuated to extend away from the collection channel and exposed. Then, upon a further positive pressure applied to the mechanical member, the collection member may be actuated to penetrate the membrane sealing the collection vessel, rendering it in fluid communication with the collection channel, thereby permitting the sample collected to flow from the collection channel to the collection vessel.

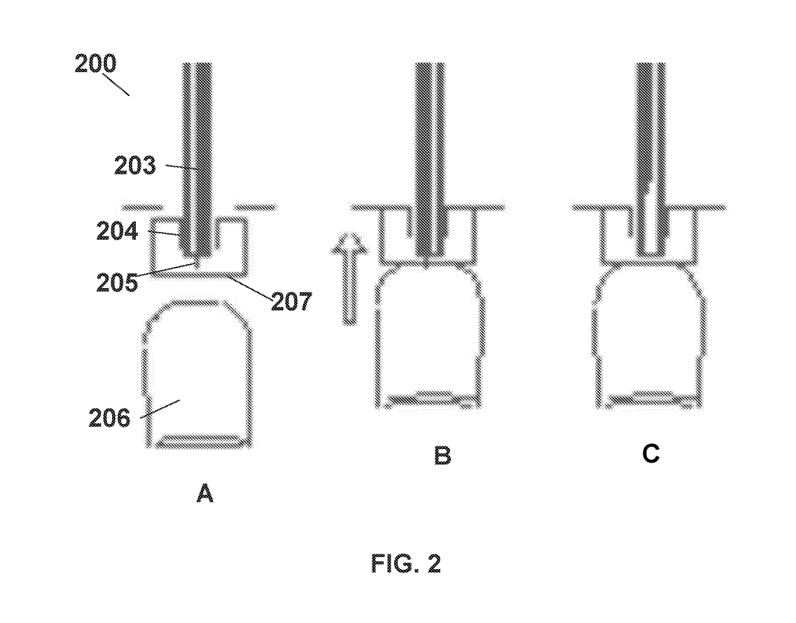

FIG. 2 shows an enlarged view of part of an exemplary collection device 200. FIGS. 2A-2C demonstrates an example of change of position of a collection member 205. FIG. 2A shows that a subject aligns his/her finger 206 with a cover 204. A collection member 205 is actuated to extend away from a collection channel 203 for collection of a sample. FIG. 2B shows that the finger moves towards the collection member, thereby also pressing the cover towards the collection member. As a result, the collection member (e.g., comprising a needle) may penetrate through the cover and may be exposed. Then, the exposed collection member may contact with and prick the finger, thereby permitting the sample (e.g., blood) to flow out of the finger. FIG. 2C shows that the collection member may then retract into the collection channel, which can be achieved by, e.g., a pressure drop generated by a finger of a user while pressing against the collection member, which may subject the sample to flow enter the collection channel (e.g., a capillary) through the opening at the end of the collection channel.

The cover 204 can further comprise a retaining member 207 (e.g., a sheet apparatus comprising a sample retaining card) on the side of the cover facing the finger. Thus, during the retraction of the collection member into the collection channel after sampling, a least a substantial part of the sample (e.g., blood) carried on the collection member may be retained (e.g., absorbed) by the retaining member. If desired, the retaining member may be used for further detection (e.g., for analyzing certain components or characteristics of the sample retained).

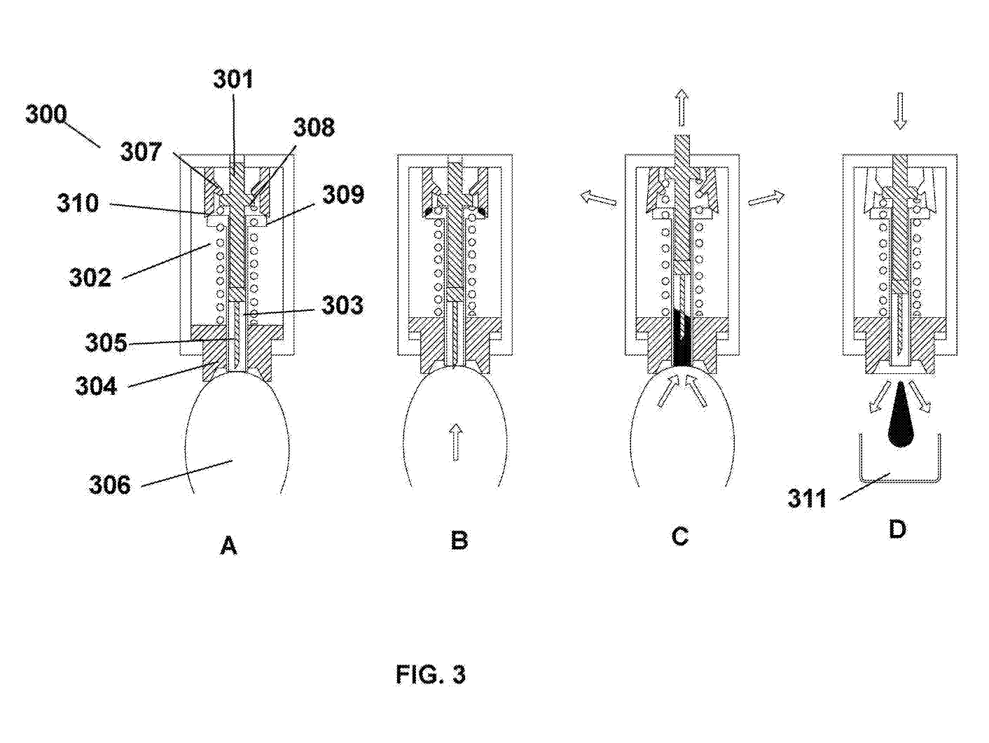

FIGS. 3A-3D demonstrates another embodiment of a sample collection device 300 of the invention. In this non-limiting example, the sample collection device 300 may include a collection device body 302, a mechanical member 301, and at least one collection channel 303 having an opening at an end thereof. The collection channel may comprise a collection member 305 at or adjacent to the end having the opening. In certain cases, the collection channel may be substantially free of any anticoagulant.

The collection device 300 can further comprise a cover 304. The cover 304 may be fitted over a portion of the collection channel 303 and over a portion of the collection member 305. The cover 304 may also be fitted within a portion of the device body 302. The cover 304 may be formed from an opaque, transparent, or translucent material. The cover 304 may be formed from a material penetrable with a collection member (e.g., a needle) and/or a collection channel (e.g., a capillary). The device body 302 may comprise one or more flanges attached to a portion thereof (e.g., an end or one or more sides thereof) adjacent to the mechanical member 301. The flanges may be present in pairs at symmetrical positions of the device body. Each of the one or more flanges may comprise at least a first contacting element 307 and a second contacting element 310. The mechanical member 301 may comprise along its length at least a first protruding element 308, wherein the first contacting element 307 may be configured to engage and be in contact with the first protruding element 308. The collection channel 303 may comprise on its outer surface (i.e., the surface facing the inner wall of the device body) at least a second protruding element 309, wherein the second contacting element 310 may be configured to engage and be in contact with the second protruding element 309. Each of the protruding elements may be present symmetrically in pairs, and the axis of symmetry may be aligned with the longitudinal axis of the mechanical member 301 and/or the collection channel 303. In some embodiments, the first and the second protruding element 308 and 309 may be positioned perpendicular to the longitudinal axis of the mechanical member 301 and the collection channel 303, respectively. In some embodiments, comparing to the first protruding element 308, the second protruding element 309 is located at a position closer to the opening of the collection channel, and the second protruding element 309 is longer than the first protruding element 308. In some embodiments, comparing to the first protruding element 308, the second protruding element 309 is located at a position closer to the opening of the collection channel, and the second protruding element 309 protrudes further away than the first protruding element 308 from their respective attaching point to the mechanical member 301 or the collection channel 303.

FIG. 3A shows that a subject aligns his/her finger 306 with the cover 304. As demonstrated in FIG. 3A, the mechanical member 301 of the device 300 is depressed at a first position, causing the collection member 305 to extend away from the collection channel 303 for collection of a sample. When the mechanical member 301 is depressed at this first position, the first contacting element 307 engages and is in contact with the first protruding element 308, thereby blocking movement of the mechanical member 301 towards an end of the device body 302 opposite to the end of the device body adjacent to the opening of the collection channel 303. Meanwhile, the second contacting element 310 is in contact with the second protruding element 309, thereby creating a pressure between the second protruding element 309 and the second contacting element 310. As such, the mechanical member 301 is fixed in the first position. The first and second contacting elements 307 and 310 may be of any shape or form suitable for engaging and/or blocking the movement of the first and the second protruding elements 308 and 309, respectively.

FIG. 3B shows that the finger moves towards the collection member, thereby also pressing the cover to move towards the opposite end of the collection channel opening. The cover may be configured to cause simultaneous movement of the collection channel, as well as movement of the second protruding element 309 comprised on the outer surface thereof. As a result, the second protruding element 309 is further pressed against the second contacting element 310, and the collection member (e.g., comprising a needle) further extends away relative to the collection channel, penetrates through the cover and is exposed. Then, the exposed collection member may contact with and prick the finger, thereby permitting the sample (e.g., blood) to flow out of the finger.

FIG. 3C shows that with increasing pressure of the second protruding element 309 against the second contacting element 310, the second contacting element 310 (and thus the portion of the flange comprising the second contacting element 310) is pushed away from the mechanical member by the second protruding element 309, causing the first protruding element 308 to be released from the blockage of the first contacting element 307, thereby enabling mechanical member 301 to move further towards the opposite direction of the collection channel opening. This results in retraction of the collection member 303 into the collection channel, allowing the sample (e.g., blood) to enter into the collection channel (e.g., a capillary) through the opening at the end of the collection channel. The movement of the sample from the finger to the collection channel can be driven by negative pressure, gravity, capillary action, aspiration, pressure differential or vacuum force, etc.

FIG. 3D shows that when sufficient sample has been collected in the collection channel, a positive pressure may be applied to the mechanical member, which then may be depressed to the first position as described above for FIG. 3A, wherein the first contacting element 307 engages and is in contact with the first protruding element 308, thereby blocking movement of the mechanical member 301 towards an end of the device body 302 opposite to the end of the device body adjacent to the opening of the collection channel 303. Meanwhile, the second contacting element 310 is in contact with the second protruding element 309, thereby creating a pressure between the second protruding element 309 and the second contacting element 310. As such, the mechanical member 301 is again fixed in the first position. In some cases, during the same process, or with a further positive pressure applied to the mechanical member, the sample collected is transferred from the collection channel through the opening to a collection vessel 311.

The collection vessel 311 is as described above in the present application. In some cases, the collection vessel 311 may comprise a membrane sealing it from its environment.

The cover 304 may further comprise a retaining member (e.g., a sheet apparatus comprising a sample retaining card) on the side of the cover facing the finger, as described above.

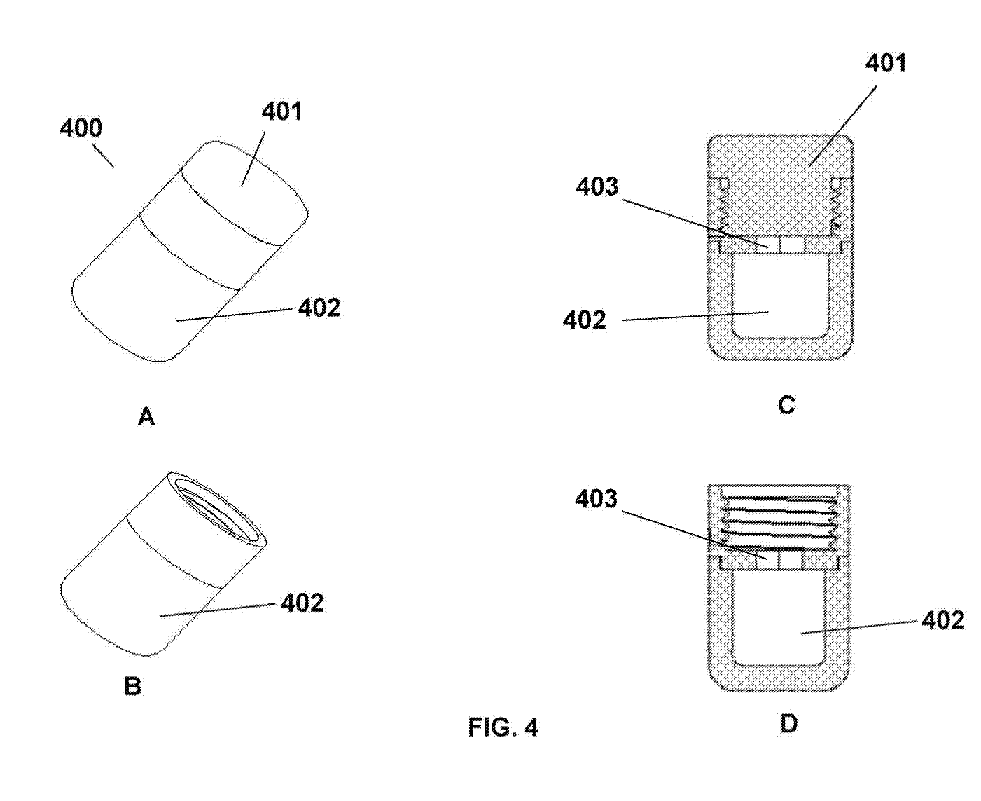

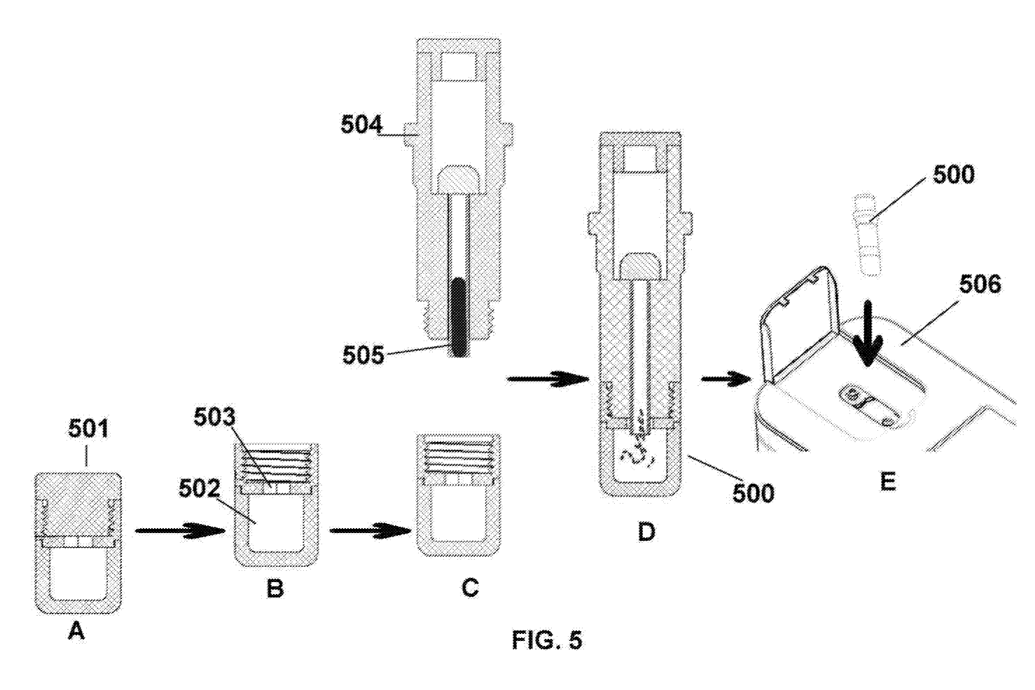

FIG. 4A provides a non-limiting example of a collection vessel 400, comprising a vessel cap 401 and a vessel body 402. FIG. 4B demonstrates a vessel body 402 without a cap. FIG. 4C shows a perspective view of the collection vessel 400, wherein the vessel body 402 comprises a membrane 403 to separate the contents (e.g., reagents necessary for nucleic acid amplification) therein from the external environment. FIG. 4D shows a perspective view of the vessel body 402 without a cap. FIG. 5 shows use of a sample collection device 500 according to one non-limiting example of the present disclosure for nucleic acid amplification. The device 500 comprises a sampling component 504 and a collection vessel, wherein the collection vessel comprises a cap 501 and a vessel body 502 containing reagents necessary for nucleic acid amplification. The vessel body 502 further comprises a membrane 503 sealing the contents (e.g., reagents necessary for nucleic acid amplification) from the environment. Before analysis, the collection vessel is in a sealed state. Immediately before use, the cap 501 is removed and separated from the vessel body 502. Then, a sample (e.g., a bodily fluid sample, e.g., a blood sample) is obtained from a subject using the sampling component 504, and the sample is retained in a collection channel 505 of the sampling component 504. Immediately or shortly after sampling, the sealing membrane 503 of the vessel body 502 is penetrated or removed, the sampling component 504 is then mounted onto the vessel body 502 to form a sealed and assembled sample collection device 500, wherein the collection channel 505 is in fluid communication with the vessel body 502 and the sample collected is released from the collection channel to the vessel body, forming a reaction mixture comprising the sample and the reagents necessary for nucleic acid amplification. The assembled device 500 is then directly placed in an appropriate apparatus 506 (e.g., a PCR machine) for amplification and further analysis. The whole process may take less than about 1 hour. A point-of care amplification device of the present disclosure can provide for fast and real-time nucleic acid amplification and pathogen detection.

### Method for Collecting a Sample of a Subject

Another aspect of the present disclosure provides methods for collecting a sample (e.g., a bodily fluid sample, e.g., a blood sample) of a subject. A method for collecting a sample from a subject can include providing a sample collection device comprising at least one collection channel. The collection channel can include a collection member having an opening at an end thereof to permit flow of the sample from a source thereof to the collection channel. Next, the opening of the collection member can be positioned adjacent to the source of the sample. The sample can then be collected from the source to the collection channel upon flow of the sample from the source through the opening into said collection channel. Next, the collected sample can be deposited from the collection channel through the opening into a collection vessel that includes reagents necessary for nucleic acid amplification. This can provide a mixture having the sample and the reagents. The mixture thus formed can be directly used for further analysis, e.g., nucleic amplification, sequencing, etc., for example, immediately after sample collection using an apparatus appropriate for performing such analysis.

In some embodiments, the collection vessel may be sealed with a membrane (e.g., a diaphragm) separating the contents (e.g., reagents necessary for nucleic acid amplification) therein from the environment. The membrane may be a synthetic membrane, e.g., a membrane formed of a solid state material (e.g., semiconductor, metal, semi-metal or non-metal) or polymeric material. For example, a membrane can be formed by an opaque, transparent, or translucent material sealing a collection vessel and separating it from the external environment. Thus, in certain embodiments, the method may further comprise the step of penetrating the membrane sealing the collection vessel, rendering it in fluid communication with the collection channel, thereby permitting the sample collected to flow from the collection channel to the collection vessel. The penetrating may be achieved with the same collection member (e.g., comprising a needle at the end) for sample collection. Alternatively, the penetrating may also be achieved with a different device or component that is able to penetrate the membrane.

The sample can be a tissue or a bodily fluid sample, or a fraction thereof. In some cases, the sample is a “bodily fluid” sample, which may include but not limited to blood, urine, saliva, tears, sweat, a bodily secretion, a bodily excretion, or any other fluid originating in or obtainable from a subject. In particular, the sample may include but not limited to blood, serum, plasma, bone marrow, saliva, urine, gastric fluid, spinal fluid, tears, stool, mucus, sweat, earwax, oil, glandular secretions, cerebral spinal fluid, semen, vaginal fluid, interstitial fluids derived from tumorous tissue, ocular fluids, placental fluid, amniotic fluid, cord blood, lymphatic fluids, cavity fluids, sputum, pus, meconium, breast milk and/or other secretions or excretions. For example, a sample can be a blood sample, or a portion thereof, which may include but not limited to a whole blood sample, a sample comprising red blood cells, plasma sample, serum sample, buffy coat sample, a sample comprising white blood cells, etc. The blood sample can be obtained directly from the subject, for example, the sample can be analyzed or tested (e.g., by amplification or sequencing) without further processing (e.g., by centrifugation, purification, etc.).

The nucleic acid amplification can be performed using the sample (e.g., the blood sample) deposited in the collection vessel. For example, the blood sample that is deposited in the collection vessel can be subjected to nucleic acid amplification conditions (e.g., PCR) without any additional processing of the blood sample (e.g., purification, centrifugation etc.).

The collection vessel can be substantially free of an anticoagulant. The reagents comprised in the collection vessel can include, but not limited to, one or more primers and one or more polymerizing enzymes. In certain cases, the reagents may comprise Mg or Mn ions.

The one or more primers may have sequences that are selected to assay for a presence or quantity of an infectious agent in said subject. The infectious agent may be associated with a disease that the subject is having, being suspected of having or at the risk of having. In some embodiments, the disease may be associated with a virus e.g., an RNA virus or a DNA virus. For example, the virus can be selected from the group consisting of human immunodeficiency virus I (HIV I), human immunodeficiency virus II (HIV II), an orthomyxovirus, Ebola virus, Dengue virus, influenza viruses, hepevirus, hepatitis A virus, hepatitis B virus, hepatitis C virus, hepatitis D virus, hepatitis E virus, hepatitis G virus, Epstein-Barr virus, mononucleosis virus, cytomegalovirus, SARS virus, West Nile Fever virus, polio virus, measles virus, herpes simplex virus, smallpox virus, adenovirus, and Varicella virus. In some embodiments, the influenza virus can be selected from the group consisting of H1N1 virus, H3N2 virus, H7N9 virus and H5N1 virus. In some embodiments, the adenovirus may be adenovirus type 55 (ADV55) or adenovirus type 7 (ADV7). In some embodiments, the hepatitis C virus may be armored RNA-hepatitis C virus (RNA-HCV). In some embodiments, the disease may be associated with a pathogenic bacterium (e.g., Mycobacterium tuberculosis) or a pathogenic protozoan (e.g., Plasmodium).

The collection vessel can be adapted to stably store the mixture comprising the sample and the reagents for a time period of at least about 10 seconds, 30 seconds, 1 minute, 5 minutes, 10 minutes, 20 minutes, 30 minutes, 40 minutes, 50 minutes, or 60 minutes. In some embodiments, the collection vessel may be adapted to stably store the mixture for a time period of at least about 1 hour, 2 hours, 3 hours, 4 hours, 5 hours, 6 hours, 12 hours, 1 day, 2 days, 3 days, 4 days, 5 days, 6 days, 1 week, 2 weeks, 3 weeks, or 1 month.

The sample may be a bodily fluid sample having a volume of no more than about 5 mL; no more than about 4 mL; no more than about 3 mL; no more than about 2 mL; no more than about 1 mL; no more than about 500 μL; no more than about 250 μL; no more than about 100 μL; no more than about 75 μL; no more than about 50 μL; no more than about 35 μL; no more than about 25 μL; no more than about 20 μL; no more than about 15 μL; no more than about 10 μL; no more than about 8 μL; no more than about 6 μL; no more than about 5 μL; no more than about 4 μL; no more than about 3 μL; no more than about 2 μL; no more than about 1 μL; no more than about 0.8 μL; no more than about 0.5 μL; no more than about 0.3 μL; no more than about 0.2 μL; no more than about 0.1 μL; no more than about 0.05 μL; or no more than about 0.01 μL. For example, the sample can have a volume of about 0.01 μL to about 5 mL, about 0.01 μL to about 4 mL, about 0.01 μL to about 3 mL, about 0.01 μL to about 2 mL, about 0.01 μL to about 1 mL, about 0.01 μL to about 0.5 μL, about 0.01 μL to about 0.4 μL, about 0.01 μL to about 0.3 μL, about 0.01 μL to about 0.2 μL, about 0.01 μL to about 0.1 μL, about 0.01 μL to about 0.05 μL.

The collection vessel can be part of the collection device. For example, the collection vessel can be grouped or packaged together with the collection device, or integrated into the collection device, e.g., in a reversible way. In some examples, the collection vessel is integrated with the collection device, and removable from the collection device.

The method as described above or a repeating unit (e.g., a cycle) thereof may be performed in a time period of less than about 1-10 minutes. In some embodiments, the time period can be less than about 5 minutes, less than about 3 minutes, less than about 1 minute, or less than about 30 seconds.

The source of the sample can be a pool of the sample in a storage vessel. The source may also be a tissue of the subject that is accessible through a puncture in the tissue. The collection member may include a needle that is adapted to generate the puncture. In some embodiment, the collection member may include a needle adjacent to a needle cover. The needle may include an opening at an end thereof. In certain embodiments, said needle can be actuatable to penetrate the needle cover for collection of the sample from the source thereof. In some embodiments, the mechanical member can be a button. The mechanical member can also be spring loaded. The mechanical member can be depressible at a first position and further depressible at a second position. Depression at the first position may cause the collection member to extend away from the collection channel for collection of the sample from a source thereof. Depression at the second position may cause the sample to flow from the collection channel to the opening. In some embodiments, depression at the second position may cause the collection channel to extend away from the body of the collection device, wherein the body includes the mechanical member. The collection vessel may include identifying information of the subject tested. The identifying information can be anonymous. In some cases, the identifying information can be on a barcode. In further embodiments, the identifying information can be in a radio-frequency identification (RFID) tag. In some embodiments, the first position and the second position may be the same position. In further embodiments, the first position and the second position may be different positions.

The sample collection device may include a mechanical member that actuates the collection member to (i) extend away from the collection channel for collection of a sample (e.g., a bodily fluid sample, e.g., a blood sample) from the source thereof, and (ii) retract into the collection channel upon collection of the sample from said source. The mechanical member can be a button. The mechanical member may be depressible at a first position and further depressible at a second position, wherein depression at the first position may cause the collection member to extend away from said collection channel for collection of the sample from the source thereof, and wherein depression at the second position may cause the sample to flow from the collection channel to the opening. In some cases, the first position and the second position are the same position, wherein depression at this position may cause the collection member to extend away from the collection channel and may also cause the sample to flow from the collection channel to the opening.

In some cases, depression at the second position may cause the collection channel to extend away from a body of the collection device, wherein the body can include the mechanical member.

The mechanical member can be (i) depressible from a first position towards a second position to extend the collection member away from the collection channel, and (ii) extendable from the second position towards the first position to retract the collection member into the collection channel upon collection of the sample.

The device can further include a cover positioned at the end of the collection channel comprising the collection member, wherein the cover is configured to prevent the opening and the collection member from being exposed to the environment and to keep them clean. The cover may further comprise a retaining member (e.g., a sheet apparatus comprising a sample retaining card, e.g., a blood card) on the side of the cover facing the subject (e.g., a finger). Thus, during the retraction of the collection member into the collection channel after sampling, a least a substantial part of the sample (e.g., blood) carried on the collection member may be retained (e.g., absorbed) by the retaining member. If desired, the retaining member may be used for further detection (e.g., analyzing certain components or characteristics of the sample retained).

The cover may be fitted over a portion of the collection channel and/or a portion of the collection member. The cover may also be fitted within a portion of the device body. The cover may be formed from an opaque, transparent, or translucent material. The cover may also be formed from a material penetrable with a collection member (e.g., a needle) and/or a collection channel (e.g., a capillary).

In one embodiment of the method, a finger (or any other part of body to be tested or sampled) of a subject is aligned with a cover or a collection member of a collection device. The mechanical member of the device is depressed at a first position, causing the collection member to extend away from the collection channel for collection of a sample. Then, the finger (or any other part of body to be tested or sampled) is moved towards the collection member, thereby also pressing the cover towards the collection member. As a result, the collection member (e.g., comprising a needle) may penetrate through the cover and may be exposed. Then, the exposed collection member may contact with and prick the finger, thereby permitting the sample (e.g., blood) to flow out of the finger. Then, the collection member may retract into the collection channel, which can be achieved by, e.g., a pressure created by the finger while pressing against the collection member, thereby the sample may enter into the collection channel (e.g., a capillary) through the opening at the end of the collection channel. Meanwhile, the mechanical member may be pushed away in a direction opposite to the opening of the collection channel. When sufficient sample has been collected in the collection channel, a positive pressure may be applied to the mechanical member (e.g., depressed to a first position), which in turn may actuate a portion of the collection channel covered by the cover and/or device body to protrude out of the cover. If desired, a further positive pressure may be applied to the mechanical member (e.g., depressed to a second position), thereby causing the sample collected to flow from the collection channel to the opening.

The sample may flow into a collection vessel, which may be capable of being in fluid communication with the collection channel. The collection vessel may comprise reagents necessary for nucleic acid amplification. Thus, the sample from the collection channel and reagents comprised in the collection vessel may form a reaction mixture. The reaction mixture can be used for further analysis.

In some embodiments, the device body may comprise one or more flanges attached to a portion thereof (e.g., an end or one or more sides thereof) adjacent to the mechanical member. The one or more flanges may be present in pairs at symmetrical positions of the device body. Each of the one or more flanges may comprise at least a first contacting element and a second contacting element. The mechanical member may comprise along its length at least a first protruding element, wherein the first contacting element may be configured to engage and be in contact with the first protruding element. The collection channel may comprise on its outer surface (i.e., the surface facing the inner wall of the device body) at least a second protruding element, wherein the second contacting element may be configured to engage and be in contact with the second protruding element.

Each of the protruding elements may be present symmetrically in pairs, with the longitudinal axis of the mechanical member and/or the collection channel to be symmetry axis. In some embodiments, the first and the second protruding element and may be positioned perpendicular to the longitudinal axis of the mechanical member and the collection channel, respectively. In some embodiments, comparing to the first protruding element, the second protruding element is located at a position closer to the opening of the collection channel, and the second protruding element is longer than the first protruding element.

In some cases, the second protruding element is located at a position closer to the opening of the collection channel than the first protruding, and the second protruding element protrudes further away than the first protruding element from their respective attaching point to the mechanical member or the collection channel.

In some examples, a finger (or any other part of body to be tested or sampled) of a subject is aligned with a cover or a collection member of a collection device provided. Then, the mechanical member of the device may be depressed at a first position, causing the collection member to extend away from the collection channel for collection of a sample. When the mechanical member is depressed at this first position, the first contacting element may engage and be in contact with the first protruding element, thereby blocking movement of the mechanical member towards an end of the device body opposite to the end of the device body adjacent to the opening of the collection channel. Meanwhile, the second contacting element may be in contact with the second protruding element, thereby creating a pressure (e.g., a counter force) between the second protruding element and the second contacting element. As such, the mechanical member will be fixed in the first position. Then, the finger (or any other part of body to be tested or sampled) may be moved towards the collection member, thereby also pressing the cover to move towards the opposite end of the collection channel opening. The cover may be configured to cause simultaneous movement of the collection channel, as well as movement of the second protruding element comprised on the outer surface thereof. As a result, the second protruding element may be further pressed against the second contacting element, and the collection member (e.g., comprising a needle) may further extend away relative to the collection channel, penetrate through the cover and be exposed. Then, the exposed collection member may contact with and prick the finger (or any other part of body to be tested or sampled), thereby permitting the sample (e.g.,, blood) to flow out. Then, with increasing pressure of the second protruding element against the second contacting element, the second contacting element (and thus the portion of the flange comprising the second contacting element) may be pushed away from the mechanical member by the second protruding element, causing the first protruding element to be released from the blockage of the first contacting element, thereby the enabling mechanical member to move further towards the opposite direction of the collection channel opening. This may result in retraction of the collection member into the collection channel, allowing the sample (e.g., blood) to enter into the collection channel (e.g., a capillary) through the opening at the end of the collection channel. The movement of the sample from the finger (or any other part of body to be tested or sampled) to the collection channel can be driven by negative pressure, gravity, capillary action, aspiration, pressure differential or vacuum force, etc. When sufficient sample has been collected in the collection channel, a positive pressure may be applied to the mechanical member, which then may be depressed to the first position as described above, wherein the first contacting element may engage and be in contact with the first protruding element, thereby blocking movement of the mechanical member towards an end of the device body opposite to the end of the device body adjacent to the opening of the collection channel. Meanwhile, the second contacting element may be in contact with the second protruding element, thereby creating a pressure (e.g., counter force) between the second protruding element and the second contacting element. As such, the mechanical member may again be fixed in the first position. During the same process, or with a further positive pressure applied to the mechanical member, the sample collected may be transferred from the collection channel through the opening to a collection vessel. In some cases, the collection vessel may comprise a membrane sealing it from its environment.

The collection vessel may be capable of being in fluid communication with the collection channel. The collection vessel may comprise reagents necessary for nucleic acid amplification. Thus, the sample from the collection channel and reagents comprised in the collection vessel may form a reaction mixture. The reaction mixture can be used for further analysis.

### Kits for Nucleic Acid Amplification

Another aspect of the present disclosure provides kits for nucleic acid amplification. A kit may comprise a collection vessel including reagents necessary for nucleic acid amplification and instructions that permit a user to use a collection device to (i) collect a sample (e.g., a bodily fluid sample, e.g., a blood sample) from a source thereof, and (ii) deposit the sample into the collection vessel to provide a mixture comprising the sample and the reagents for nucleic acid amplification in a time period that is less than about 1 hour, e.g., less than about 50 minutes, less than about 40 minutes, less than about 30 minutes, less than about 20 minutes, less than about 15 minutes, less than about 10 minutes, less than about 5 minutes, less than about 4 minutes, less than about 3 minutes, less than about 2 minutes, less than about 1 minute, less than about 50 seconds, less than about 40 seconds, less than about 30 seconds, less than about 20 seconds, or less than about 10 seconds. For example, the time period can be about 10-30 seconds, in about 1-5 minutes, in about 1-10 minutes, in about 1-15 minutes, in about 1-20 minutes, in about 1-30 minutes, in about 1-40 minutes, in about 1-50 minutes or in about 1-60 minutes. The collection vessel may be substantially free of any anticoagulant.

The instructions can permit the user to perform nucleic acid amplification using the sample. The instructions can be in textual form, graphical form, or textual and graphical form. The instructions can be in a physical medium (e.g., paper) or an electronic medium (e.g., computer memory). The instructions can include one or more operations that a user may follow to perform nucleic acid amplification using the sample.

The kit can further include a collection device, as described elsewhere herein. The collection device can include at least one collection channel that includes a collection member having an opening at an end thereof to permit flow of the sample from a source thereof to the collection channel.

The kit can include a package or container. The kit can include a box (e.g., recyclable box) that includes a collection device, reagents for nucleic acid amplification and instructions. The kit can include identifying information, which can permit the kit to be identified and/or associated with a user. In some cases, the identifying information permits the kit to be anonymously associated with the user. The identifying information can be a barcode or a radio-frequency identification (RFID) tag.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A device for collecting a bodily fluid sample of a subject for nucleic acid amplification, comprising:
a device body that is coupled to at least one collection channel;
a collection member having an opening at an end thereof to permit flow of said bodily fluid sample from a source of said bodily fluid sample to said collection channel, wherein said collection channel is substantially free of an anticoagulant; and
a mechanical member coupled to said device body, wherein said mechanical member actuates said collection member to (i) extend away from said collection channel for collection of said bodily fluid sample from said source of said sample, and (ii) retract into said collection channel upon collection of said bodily fluid sample from said source.

2. (canceled)

3. (canceled)

4. The device of claim 1, wherein said collection channel is substantially contained in said body.

5. The device of claim 1, wherein said mechanical member is substantially contained in said body.

6. The device of claim 1, further comprising a collection vessel capable of being in fluid communication with said collection channel, wherein said collection vessel includes reagents necessary for nucleic acid amplification.

7. The device of claim 6, wherein said collection vessel is substantially free of an anticoagulant.

8. The device of claim 6, wherein said reagents include one or more primers and a polymerizing enzyme.

9. The device of claim 6, wherein said reagents include magnesium (Mg) or manganese (Mn) ions.

10. The device of claim 8, wherein said one or more primers have sequences that are configured to assay for a presence of an infectious disease in said subject.

11. The device of claim 6, wherein said collection vessel is adapted to stably store said bodily fluid sample for a time period of at least about 5 minutes.

12. (canceled)

13. The device of claim 1, wherein said source is a pool of said bodily fluid sample in a storage vessel.

14. (canceled)

15. The device of claim 1, wherein said end includes a needle that is adapted to puncture a tissue to collect said bodily fluid sample.

16. The device of claim 1, wherein said collection member includes a needle adjacent to a needle cover, wherein said needle includes said opening, and wherein said needle is actuatable to penetrate said needle cover for collection of said bodily fluid sample from said source.

17. The device of claim 1, wherein said mechanical member is a button.

18. The device of claim 1, wherein said mechanical member is spring loaded.

19. The device of clam 1, wherein said mechanical member is depressable at a first position and depressable at a second position, wherein depression at said first position causes said collection member to extend away from said collection channel for collection of said bodily fluid sample from said source of said sample, and wherein depression at said second position causes said bodily fluid sample, when resident in said collection channel, to flow out of said collection channel.

20. The device of claim 19, wherein depression at said second position causes said collection channel to extend away from a body of said collection device, wherein said body includes said mechanical member.

21. The device of claim 6, wherein said collection vessel includes identifying information of said subject.

22. The device of claim 6, wherein said collection vessel includes identifying information that anonymously identifies said bodily fluid sample with said subject.

23. The device of claim 21, wherein said identifying information is encoded in a barcode.

24. The device of claim 21, wherein said identifying information is encoded in a radio-frequency identification (RFID) tag.

25. A method for collecting a bodily fluid sample of a subject, comprising:
(a) providing a sample collection device comprising at least one collection channel, wherein said collection channel includes a collection member having an opening at an end thereof to permit flow of said bodily fluid sample from a source of said bodily fluid sample to said collection channel;
(b) positioning said opening of said collection member adjacent to said source of said sample;
(c) collecting said bodily fluid sample from said source to said collection channel upon flow of said bodily fluid sample from said source through said opening into said collection channel; and
(d) depositing said bodily fluid sample collected in (c) from said collection channel through said opening into a collection vessel that includes reagents necessary for nucleic acid amplification, thereby providing a mixture having said bodily fluid sample and said reagents.

26.-48. (canceled)

49. A kit for nucleic acid amplification, comprising:
a collection vessel including reagents necessary for nucleic acid amplification, wherein said collection vessel is substantially free of an anticoagulant; and
instructions that permit a user to use a collection device to (i) collect a bodily fluid sample from a source of said sample, and (ii) deposit said bodily fluid sample into said collection vessel to provide a mixture comprising said bodily fluid sample and said reagents for nucleic acid amplification in a time period that is less than about 1 minute.

50.-53. (canceled)
